(19) United States
(12) Patent Application Publication
(10) Pub. No.: US 2009/0312665 A1
(43) Pub. Date: Dec. 17, 2009

(54) MENTAL WORK LOAD DETECTOR AND MOTORCYCLE INCLUDING THE SAME

(75) Inventors: Hiroshi Daimoto, Shizuoka (JP); Akihiro Yagi, Hyogo (JP)

Correspondence Address: YAMAHA HATSUDOKI KABUSHIKI KAISHA;C/O KEATING & BENNETT, LLP, 1800 Alexander Bell Drive, SUITE 200, Reston, VA, 20191, US

(73) Assignee: YAMAHA HATSUDOKI KABUSHIKI KAISHA, Iwata-shi (JP)

(21) Appl. No.: 12/469,981

(22) Filed: May 21, 2009

(30) Foreign Application Priority Data
Jun. 11, 2008 (JP) JP2008-152641

(51) Int. Cl. A61B 5/0476

(52) U.S. Cl. 600/544

(57) ABSTRACT

A mental work load detector arranged to sense the electroencephalogram of a person and estimate a mental work load on the perceptual-motor system and a mental work load on the perceptual-central processing system separately from each other includes a electroencephalogram sensor arranged to sense the electroencephalogram of a rider, an eye fixation-related potential detector arranged to detect an eye fixation-related potential by analyzing the electroencephalogram and to detect a lambda response, an event-related potential measuring device arranged to measure an event-related potential in response to a sound from a speaker and to detect auditory p300, a lambda response comparator arranged to determine whether the lambda response has decreased, an auditory p300 comparator arranged to determine whether the auditory p300 has decreased, and a mental work load determiner arranged to determine that the mental work load on the perceptual-central processing system has increased if the lambda reaction and the auditory p300 has decreased and that the mental work load on the perceptual-central processing system has increased if the auditory p300 has decreased but the lambda response has not decreased.

### BACKGROUND OF THE PRESENT INVENTION

1. Field of the Present Invention

The present invention relates to a mental work load detector and a motorcycle including the mental work load detector, and more specifically, to a mental work load detector that senses the electroencephalogram of a person to measure an event-related potential such as an eye fixation-related potential and thus estimates loads on the perceptual-motor system (also referred to as “visual-operation system”) and the perceptual-central processing system (also referred to as “thinking system”) and a motorcycle including the mental work load detector.

2. Description of the Background Art

With the advent of advanced information communication technology in recent years, people have had easier access to various kinds of information. However, the capacity that can be handled by the user of information equipment is limited and the usability drops when the capacity is exceeded. Assuming that a person rides a motorcycle, the rider as the user operates the “handle,” “accelerator,” “brake,” and “shift-change,” while visually recognizing a “road environment” (including other vehicles, pedestrians, signs, and the like), the “speedometer,” or a “navigation system.” An excessive amount of information could lead to misidentification, misjudgments or erroneous operation. When a new information providing device is to be developed, such a device should take account into its effect on information processing by the user. To this end, indexes used to evaluate the load level of information processing by the user are necessary.

When reference is made to a human information processing process during riding (driving) activities, the process is often divided into “cognition,” “judgment,” and “operation.” The cognition is a step used to input information necessary for judgment while the user pays attention to environmental sounds or in-vehicle noises and appropriately directs the line of sight about the road environment. The judgment is a step used to determine how to react to the input information based on the user's experiences and knowledge. The operation is a step used to carry out the determined reaction, which may include automated riding actions independent of the judgment step. During the information processing process including the cognition, the judgment, and then the operation that requires determination, a mental work load is imposed on the perceptual-central processing system. During the information processing process including the cognition and then operation that does not require determination, a mental work load is imposed on the perceptual-motor system.

In order to evaluate the load level of information processing by the user in real time, a thinking-aloud method used for usability evaluation may be employed. However, the method is a qualitative evaluation and hardly applicable to an activity automated based on a perceptual-motor reaction and an activity that lasts only briefly until a problem is solved. For real-time quantitative evaluation, the line of sight or biological reactions must be measured using an eye camera or by biometric engineering. However, if the line of sight is turned toward a certain object, the information is not always processed correctly. An information processing process of a biological reaction by a circulatory system such as heart rate can be estimated only indirectly. More specifically, an evaluation index that directly reflects the information processing process carried out in the brain is necessary.

An event-related potential (ERP) is a kind of electroencephalogram and represents a brain potential related to an external or internal event. The event-related potential is an index that directly reflects information processing process in the brain, and therefore it is believed that a mental process that is not apparent from expressive behaviors measurable using an eye camera or the like can be analyzed using it.

The event-related potentials are divided into a lambda response for an eye fixation-related potential (EFRP) and auditory p300 that have been reported as possible indexes for mental work loads. It has been known that the eye fixation-related potential is a kind of event-related potential derived from the back of the head (Oz) with respect to the ending point of a saccadic eye movement (generally referred to as “saccade” or “saccades”) as a reference, and a relatively large positive component (generally referred to as “lambda response”) appears about 100 ms after the reference point. It has been known that during a saccade, visual information processing is restricted, and therefore, the lambda response would reflect that the visual information is obtained. It has been pointed out that the lambda response is originated from the primary visual area that is the same as the p100 component of a visual evoked potential. According to conventional studies, it has been reported that the peak amplitude of a lambda response is lowered by a mental work load. Consequently, when a mental work load affects the process of obtaining visual information, the amplitude of the lambda response would be lowered.

On the other hand, according to another study, a mental work load during a visual task is evaluated using an acoustic event-related potential p300 (hereinafter referred to as “auditory p300”). More specifically, a subject is required to carry out a visual task as a primary task and react to an acoustic stimulus as a secondary task according to a dual task technique. As the primary task becomes more difficult, the amount of processing resources directed thereto increases, and therefore the amount of processing resources directed to the secondary task decreases. As the result, it has been reported that the amplitude of the auditory p300 with respect to the sound of the secondary task decreases. The auditory p300 represents a plurality of acoustic stimuli having different occurrence rates and is a positive component that appears around 300 ms after the point where a target event is presented when a subject is requested to carry out a button pressing reaction to an acoustic stimulus with a low occurrence rate as the target. It is believed that the auditory p300 in response to a target stimulus is distributed across the top of the head (Pz), reflects the amount of distribution of the processing resources in the perceptual-central processing level, and is related to the end of the cognition coding processing or updating of an operation memory. From the foregoing, as the processing resources that can be utilized are reduced because of the mental work load for the primary task, the amplitude of the auditory p300 for the secondary task would decrease. It was reported in past studies that the lambda response and the auditory p300 both had their amplitudes lowered by a mental work load. However, it has not been clear how these indexes change when the lambda response and the auditory p300 are measured simultaneously under mental work loads with different qualities.

JP-A 2007-125184 discloses an eye fixation-related potential analyzer that can highly precisely evaluate the degree of attention concentration by classifying saccades depending on various states and calculating eye fixation-related potentials. However, only the overall degree of attention concentration becomes available with the device. If, for example, the degree of attention concentration of a rider that rides a motorcycle is evaluated, it cannot be determined whether the rider concentrates his/her attention on the riding operation (such as handling, accelerating, braking, and shift-changing, which corresponds to a load on the perceptual-motor system) or on displays made by information equipment (such as a speedometer and a navigation system) and the surrounding environment (such as other vehicles, pedestrians, and signs).

JP-A 2002-272693 discloses an eye fixation-related potential analyzer that can detect eye fixation-related potentials at a plurality of sites of a head and map the state of the brain activities, so that the degree of attention concentration can be evaluated using the eye fixation-related potentials. However, although the analyzer allows a site dominant in the activities to be determined, it cannot be determined whether the rider concentrates his/her attention on the riding operation or displays by information equipment or the surrounding environment by evaluating the degree of concentration attention of the rider riding the motorcycle using the analyzer.

JP-A 2007-052601 discloses a usability evaluator that can evaluate how readily a user can learn various functions of a device in use, how quickly the user can become accustomed to the device, and the degree of the user's interest using event-related potentials. It can be determined whether the state of information processing carried out by the user while the user operates the equipment (riding or recognizing information equipment and the environment), but it was found from experimental studies that the state could change even for information processing necessary for the riding operation. For example, if the degree of attention concentration of a rider riding a motorcycle is evaluated using the evaluator, it cannot be determined whether the rider concentrates his/her attention on the riding operation or the displays of information equipment or the surrounding environment.

### SUMMARY OF THE INVENTION

In order to solve the above-described problems, preferred embodiments of the present invention provide a mental work load detector arranged to sense the electroencephalogram of a person and estimate the kind and load of a mental work.

A mental work load detector according to a preferred embodiment of the present invention includes an electroencephalogram sensor, first and second biological reaction detectors, first and second biological reaction comparators, and a mental work load estimator.

The electroencephalogram sensor senses the electroencephalogram of a person. The first biological reaction detector is arranged to detect in time series a first biological reaction related to a first mental work load by analyzing the electroencephalogram sensed by the electroencephalogram sensor. The second biological reaction detector is arranged to detect in time series a second biological reaction related to a second mental work load different in quality from the first mental work load by analyzing the electroencephalogram sensed by the electroencephalogram sensor. The first biological reaction comparator is arranged to detect whether the first biological reaction detected by the first biological reaction detector has changed in time series. The second biological reaction comparator determines whether the second biological reaction detected by the second biological reaction detector has changed in time series. The mental work load estimator is arranged to estimate the kind and load of the mental work imposed on the person based on the result of determination by the first and second biological reaction comparators.

According to a preferred embodiment of the present invention, at least two kinds of biological reactions that are different in quality are preferably detected from the electroencephalogram of a person, and therefore the kind and load of a mental work can be estimated based on whether each of the biological reactions has changed.

The mental work load detector may preferably further include a sensory stimulator arranged to provide the person with a sensory stimulus. The first biological reaction detector may include, for example, an eye fixation-related potential measuring device arranged to measure an eye fixation-related potential. The second biological reaction detector may include, for example, an event-related potential measuring device arranged to measure an event-related potential in response to the stimulus provided by the sensory stimulator.

The stimulus provided by the sensory stimulator may preferably be an acoustic stimulus. The eye fixation-related potential measuring device may include, for example, a saccade detector and a lambda response detector. The saccade detector is arranged to detect the saccade of the person. The lambda response detector is arranged to detect a lambda response as the first biological reaction in response to the saccade detected by the saccade detector. The event-related potential measuring device may include, for example, an auditory p300 detector arranged to detect an auditory p300 as the second biological reaction in response to the acoustic stimulus provided by the sensory stimulator.

The first biological reaction comparator may preferably include a lambda response comparator arranged to determine whether the lambda response detected by the lambda response detector has decreased. The second biological reaction comparator may include, for example, an auditory p300 comparator arranged to determine whether the auditory p300 detected by the auditory p300 detector has decreased. The mental work load estimator may include, for example, a determiner arranged to determine that the mental work loads on the perceptual-motor system and the perceptual-central processing system of a person have increased if the auditory p300 comparator determines that the auditory p300 has decreased and the lambda response comparator determines that the lambda response has decreased and a determiner arranged to determine that the mental work load on the perceptual-central processing system has increased if the auditory p300 comparator determines that the auditory p300 has decreased and the lambda response comparator determines that the lambda response has not decreased.

In this way, the mental work load on the perceptual-motor system and the mental work load on the perceptual-central processing system of the person can separately be estimated.

The mental work load detector may preferably further include an operation input device arranged to receive the operation of the person. The first biological reaction detector may include, for example, an eye fixation-related potential measuring device arranged to measure an eye fixation-related potential. The second biological reaction detector may include, for example, an event-related potential measuring device arranged to measure an event-related potential in response to the operation received by the operation input device.

In this way, the event-related potential in response to the input operation is measured, and therefore the above-described sensory stimulator is not necessary.

The mental work load detector may preferably further include a response content determiner and an equipment responder. The response content determiner is arranged to determine the content or timing of information perceived by the person that is to be presented by equipment according to the kind and load of the mental work estimated by the mental work load estimator. The equipment responder is arranged to present the person with the information based on the content or in the timing determined by the response content determiner.

In this way, the person can be presented with information based on an appropriate content or at an appropriate time.

Alternatively, the response content determiner determines the operation mode of the equipment operated by the person according to the kind and load of the mental work estimated by the mental work load estimator. The equipment responder is arranged to set the equipment to the operation mode determined by the response content determiner.

In this way, the person can operate the equipment in an appropriate operation mode.

Other features, elements, steps, characteristics and advantages of the present invention will become more apparent from the following detailed description of preferred embodiments of the present invention with reference to the attached drawings.

### BRIEF DESCRIPTION OF THE DRAWINGS

FIG. 1 is a front view of a display illustrating a tracking task used in a verification experiment according to a preferred embodiment of the present invention.

FIG. 2A is a graph showing saccades and eye fixation-related potentials included in an electroencephalogram.

FIG. 2B is a graph showing portion IIB in FIG. 2A enlarged.

FIG. 3A is a graph showing a target event and an event-related potential included in an electroencephalogram.

FIG. 3B is a graph showing portion IIIB in FIG. 3A enlarged.

FIG. 4 is a graph showing a lambda response detected from an eye fixation-related potential in a verification experiment according to a preferred embodiment of the present invention.

FIG. 5 is a graph showing the average of the peak amplitude of a lambda response in each condition for a mental work load and the standard deviation.

FIG. 6 is a graph showing auditory p300 detected from an event-related potential in a verification experiment according to a preferred embodiment of the present invention.

FIG. 7 is a graph showing the average of the peak amplitude of auditory p300 in each condition for a mental work load and the standard deviation.

FIG. 8 is a side view of the structure of a motorcycle including a mental work load detector according to a preferred embodiment of the present invention.

FIG. 9 is a functional block diagram showing the configuration of a mental work load detector according to a first preferred embodiment of the present invention.

FIG. 10 is a flow chart for illustrating a program that controls the mental work load detector shown in FIG. 9 or its operation.

FIG. 11 is a flow chart for illustrating details of the process from comparison to response content determination shown in FIG. 10.

FIG. 12 is a functional block diagram showing the configuration of a mental work load detector according to a second preferred embodiment of the present invention.

### DETAILED DESCRIPTION OF THE PREFERRED EMBODIMENTS

Preferred embodiments of the present invention will now be described in detail with reference to the accompanying drawings, in which the same or corresponding portions are denoted by the same reference characters, and their description will not be repeated.

### Verification Experiments

### 1. Method

Before describing preferred embodiments of the present invention, verification experiments that led to the findings on which preferred embodiments of the present invention are based will be described in detail. In the experiments, the lambda response and the auditory p300 were both measured at the same time during multiple tracking tasks and the effects of two kinds of mental work loads, in other words, the load on the perceptual-motor system and the perceptual-central processing system of a person, on these indexes were verified.

### 1.1 Subjects

The subjects were right-handed college and graduate school students and workers, 18 people altogether including ten men and eight women, who gave informed consents and their average age was 21.9. They all had normal eyesight (or corrected eyesight) and normal hearing ability.

### 1.2 Tasks

The subjects were requested to carry out three kinds of tasks at the same time (a tracking task, a numerical task, and an oddball task). However, some of the task requests did not include any numerical task. The period for performing the task was 5 minutes. According to the priority order of the tasks, the tracking task was the first, the numerical task was the second, and the oddball task was the third in the order. A subject was requested to carry out the tasks in the order of priority as correctly and quickly as possible. The luminance in the lab was kept at 72 Lx.

### 1.2.1 Tracking Tasks

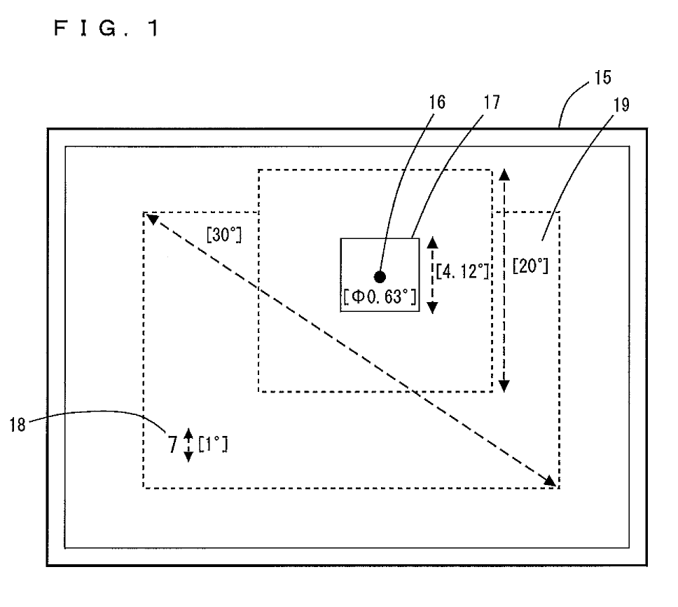

The tracking task was set as a load task for the perceptual-motor system. More specifically, as shown in FIG. 1, a subject was requested to move a tracking frame 17 with a track ball so that a target 16 (φ=0.63°, black) moving randomly at about 2.51°/s on the positive screen (white) of a 100 inch display 15 did not move out from the tracking frame 17. The degree of difficulty of the tracking task was controlled based on the presence/absence of a change in the speed of the target 16. When there was no change in the speed of the target 16, the moving speed of the target 16 was fixed at about 2.51°/s. When there was a change in the speed, the speed was raised to about 5.6°/s in synchronization with the period in which a single digit number 18 from “0” to “9” used for a numerical task was put up on the screen. The subject was seated on a chair and lightly pressed his/her chin against a face positioner so that the visual recognition distance from the display 15 was about 274 cm. The track ball was controlled by the right hand.

### 1.2.2 Numerical Tasks

The numerical task was set as a load task for the perceptual-central processing system. More specifically, when the single digit numbers 18 were randomly presented on the same screen as that for the tracking operation, a subject was requested to simply read aloud the single digit numbers 18 or add them. For the addition, the subject was requested to read aloud only the one's digit of the result produced by adding the “immediately previously presented number” to the “presently presented number.” The number presented for the first time had no previous number and therefore the number was simply read aloud. The numbers were each presented for 1.0 s at the intervals of 2.0 S. (The number of single digit numbers presented for five minutes=100). The single digit numbers 18 were presented in a peripheral viewing field region, in other words, in the peripheral portion 19 of the target 16. The distance from the target 16 to the single digit number 18 was 10° at a minimum and 30° at a maximum. The single digit numbers 18 were presented in random positions in the peripheral portion 19 and their size was about 1°.

### 1.2.3 Oddball Tasks

The oddball task was set as a task used to determine an auditory p300. More specifically, the task was a three-sound oddball task. Three pure tones, at 1800 Hz (p=0.70, standard stimulus), 2000 Hz (p=0.15, target event), and 500 Hz (p=0.15, deviant stimulus) were used as a probe stimulus. The duration was 70 ms, and the rise and fall time was 10 ms. These stimuli were presented in a random order at about 60 dB SPL from a headset. The target event and the deviant stimulus were prevented from being continuously presented. The onset interval for the stimuli was set to 700 ms in order to detect as many triggers as possible for the five-minute task time. The target event was presented 67 times during the five-minute task. Each subject paid attention to the presentation of the target events and contacted the index finger or thumb of the left hand against a pressure sensor.

### 1.3 Protocol

There were six conditions for experiments as given in the following Table 1 and they were combinations of a tracking task (a mental load on the perceptual-motor system) and a numerical task (a mental load on the perceptual-central processing system).

TABLE 1

| experiment | tracking task | |
|---|---|---|
| condition | (speed change) | numerical task |
| EN | none | none |
| EE | none | reading aloud |
| ED | none | addition |
| DN | present | none |
| DE | present | reading aloud |
| DD | present | addition |

In condition EN (Easy tracking and Non-numerical task), there was no change in the tracking speed and no presentation of numbers. In condition EE (Easy tracking and Easy numerical task), there was no change in the tracking speed, single digit numbers were presented, and the subjects were requested to read them aloud. In condition ED (Easy tracking and Difficult numerical task), there was no change in the tracking speed, single digit numbers were presented, and the subjects were requested to carry out addition. In condition DN (Difficult tracking and Non-numerical task), there was change in the tracking speed, and no number was presented. In condition DE (Difficult tracking and Easy numerical task), there was a change in the tracking speed, single digit numbers were presented, and the subjects were requested to read them aloud. In condition DD (Difficult tracking and Difficult numerical task), there was a change in the tracking speed, single digit numbers were presented, and the subjects were requested to carry out addition. In the oddball task, the subjects were requested to respond to target stimuli in all of the conditions. The subjects carried out six kinds of tracking operations for five minutes each, in other words, they carried out the tracking tasks for 30 minutes in total. The subjects answered questionnaires immediately after the performance in each condition and took a one-minute break between each condition. After the end of the performance in all the conditions, the subjects reported about which target condition was difficult and the subjective order of the tasks. Six orders of testing for the conditions were set for counterbalancing. Before the experiments, the subjects practiced each task for five minutes in total.

### 1.4 Measurement

Electroencephalograms and eye potentials were measured while the tasks in all the conditions were carried out. The electroencephalograms (EEG) were derived with reference to linked earlobes using a silver-silver chloride electrode. There were four sites altogether for derivation according to International 10-20 electrode layout method, i.e., the front portion of the head (Fz), the central portion (Cz), the top of the head (Pz), and the back of the head (Oz) on the midline. The lower cut-off frequency was 0.80 Hz and the higher cut off frequency was 30 Hz. The electrooculogram (EOG) was derived from a pair of silver-silver chloride electrodes mounted outside binocular fissures to detect horizontal saccades and another pair mounted at the upper and lower edge of the left eye socket to detect vertical saccades and blinking, in other words, bipolar derivation was carried out. The ground electrode was placed at the forehead. Similarly to the electroencephalograms, the lower cut-off frequency was 0.80 Hz and the higher cut off frequency was 30 Hz. These physiological responses were all measured using a bio-amplifier and recorded on a hard disk. The sampling frequency was 500 Hz. The electrode impedance was not more than 10 kΩ. The behavioral value was measured for each of the tracking tasks, the numerical tasks, and the oddball tasks. In the tracking task, the number of times that the tracking frame 17 was missed was measured as a tracking error. The tracking error was the number of times a target 2 deviated by at least one dot for more than 100 ms. The ordinates of the target 2 were measured for each 10 ms. In the numerical task, the face of a subject was taken using a video camera and recorded with voice sound for reading aloud numbers and the result of addition. The recorded voice sound was compared to a presented number output as the log file of the tracking program and determined for correctness. An erroneous speech was counted as an utterance error. In the oddball task, the “timing for presenting three sounds for probe stimulation,” and “the presence/absence of a subject's response” were recorded at a sampling frequency of 500 Hz on the same hard disk that recorded the electroencephalograms and eye potentials. A correct response to a target event was counted as a target event response.

### 1.5 Questionnaires

In the questionnaires, the subjective degrees of attention concentration or fatigue immediately after the performance in each of the conditions were subjectively evaluated. The evaluation was carried out by a 7-point rating scale. Furthermore, after all the tracking tasks and answers to the questionnaires ended, the subjects were requested to answer the order based on the degrees of difficulty of the conditions.

### 2. Analyzing Event-Related Potentials

### 2.1 Eye Fixation-Related Potentials

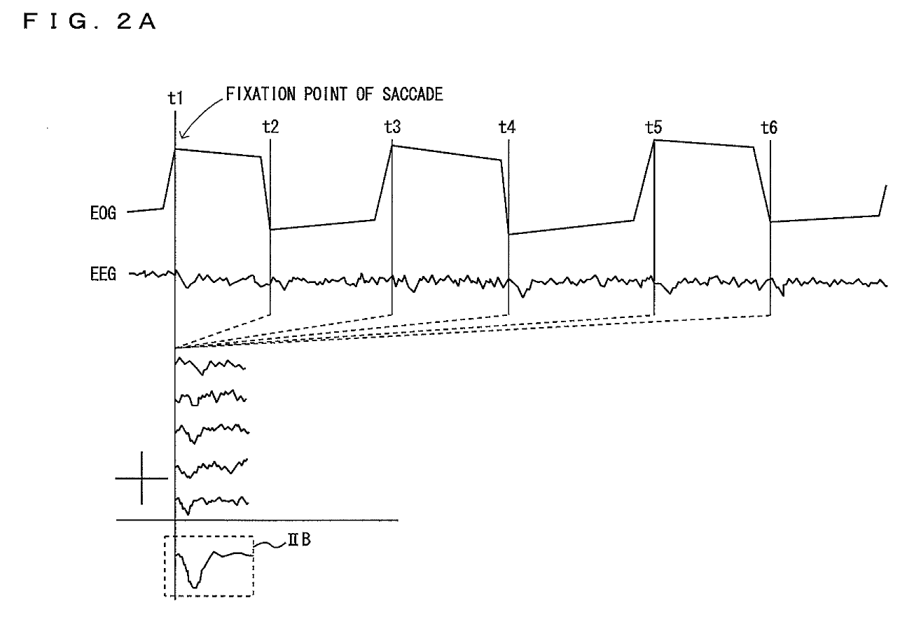

The eye fixation-related potential was analyzed with reference to the back of the head (Oz) which is a lambda response-dominant site. As shown in FIGS. 2A and 2B, saccade ending points t1 to t6 were detected from the horizontal eye movement EOG, and averaging processing was carried out to the electroencephalogram EGG using the time points as triggers. When an artifact such as blinking was included, addition was not carried out. In conditions EN and DN, numbers were not presented in the peripheral viewing field region and therefore there was almost no saccade. Saccades were generated in conditions EE, ED, DE, and DD in which numbers were presented, and the average was 160.35 times, which was a sufficient number of saccades (EE: 143.66 times, ED: 142.56 times, DE: 197.39 times, and DD: 157.78 times). The averaging waveform (shown in FIG. 2B) was obtained by producing an arithmetic mean for each condition during a section of 700 ms in total from 200 ms before the end of each saccade and 500 ms after the end of the saccade. The peak amplitude of a positive component (lambda response) appearing at the point of about 100 ms in the waveform thus obtained was calculated. The base line was set equal to the average potential from 100 ms to 200 ms before the end of the saccades. Note that no saccade appeared in conditions EN and DN in which no number was presented and these were excluded from the analysis.

2.2 Auditory p300

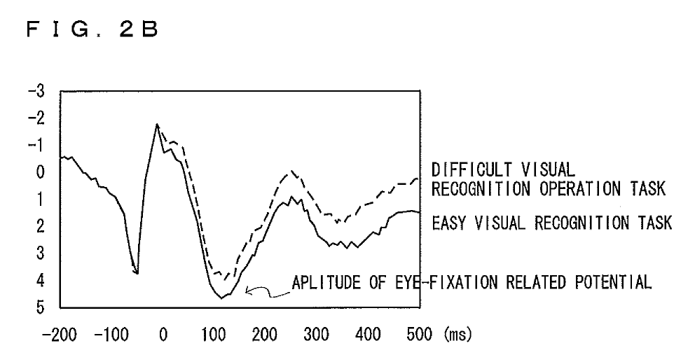

As for the auditory p300, the top of the head (Pz) as a P300 dominant site in response to a target event was analyzed. As shown in FIGS. 3A and 3B, the auditory p300 was detected as triggers for arithmetic means set at the points of the target event presentation. The arithmetic mean waveform was obtained for each condition by producing an arithmetic mean for a section of 900 ms in total from 200 ms before the presentation of each event to 700 ms after the event. During the analysis of auditory p300, when an artifact such as a blinking and an eyeball movement was included, an addition was not carried out. The number of trials that can be used for the arithmetic mean processing was less than that for eye fixation-related potentials, and the number was 41.15 times on average (from 31.39 times to 55.61 times) for each condition. When the number of additions is small and the number is different among the conditions, the S/N ratio must be taken into account and therefore, the arithmetic mean processing was carried out according to the condition with the least addition number for each subject. When the number of additions was more than sufficient, the arithmetic mean processing was carried out to the median portion of addition data candidates obtained from the trials in the five-minute period. The number of additions used for detecting the auditory p300 was eventually different among the subjects, the minimum number was 17 and the maximum number was 42. As a result, the average of the number of additions in each condition was all 28. The peak amplitude of a positive component (auditory p300) appearing from 200 ms to 600 ms in the waveform thus obtained was calculated. The baseline was set equal to the average potential in the 200 ms period before the presentation of the event.

### 3. Results

### 3.1 Subjective and Behavioral Measures

Subjective and behavioral measures in the conditions are listed in the following Table 2.

TABLE 2

| | EN | EE | ED | DN | DE | DD |
|---|---|---|---|---|---|---|
| difficulty (order) | 6.00 | 4.39 | 2.28 | 4.61 | 2.61 | 1.11 |
| attention | 4.11 | 4.78 | 5.22 | 4.22 | 5.17 | 5.72 |
| (min: 1 to max: 7) | | | | | | |
| fatigue | 3.50 | 3.94 | 4.11 | 4.00 | 4.06 | 4.56 |
| (min: 1 to max: 7) | | | | | | |
| tracking error | 26.06 | 25.94 | 27.89 | 151.28 | 152.50 | 139.22 |
| (number of times) | | | | | | |
| utterance error | — | 1.68 | 18.91 | — | 3.25 | 21.27 |
| (number of times) | | | | | | |
| target event response | 46.22 | 31.06 | 18.56 | 42.11 | 25.83 | 17.00 |
| (number of times) | | | | | | |

As for the order of the degree of subjective difficulty, the Wilcoxon's signed rank sum test (Holm correction) was carried out. Note that according to the various preferred embodiments of the present invention, Type 1 errors that could be caused by the multiplicity of testing were taken into consideration, the degree of freedom was corrected by the Holm correction method, and the significant level was set to 5%. Therefore, a significant difference was observed at 99-percent confidence level in all the combinations other than in the combination of ED and DE and the combination of EE and DN. In other words, as for the order in the degree of subjective difficulty, condition DD was in the first place, conditions ED and DE in the second place, conditions EE and DN in the third place, and condition EN in the fourth place.

For the degree of attention concentration, the degree of fatigue, the number of tracking errors, the number of utterance errors, and the number of target event responses, analysis of variance (ANOVA) was applied to a two-factor within-subject design for a tracking task (two levels) and a numerical task (three levels). As a result, as for the degree of attention concentration, a main effect was observed in the numerical task (F(1.35, 22.87)=17.08, p<0.01). A t-test (Holm correction) as a corresponding sub-effect test was carried out and a significant difference was observed at 99-percent confidence level for all the combinations of the three levels. More specifically, the degree of attention concentration had higher values in proportion to the degree of difficulty of the numerical tasks.

For the degree of fatigue, a main effect was observed in the numerical task (F(1.73, 29.32)=4.73, p<0.05). Then, a t-test (Holm correction) as a corresponding sub-effect test was carried out, and a significant difference was observed at 99-percent confidence level between the addition tasks (ED, DD) and no task (EN, DN). More specifically, the degree of fatigue was increased by the addition task.

For the number of tracking errors, a main effect was observed in the tracking task (F(1, 17)=449.92, P<0.01). More specifically, the number of tracking errors increased as the degree of tracking difficulty increased.

For the number of utterance errors, a main effect was observed in the numerical task (F(1, 17)=45.65, p<0.01). More specifically, the number of utterance errors increased by the addition task.

For the number of target effect responses, a main effect was observed both in the tracking task and the numerical task (the tracking task: F(1, 17)=13.00, p<0.01, the numerical task: F(1.44, 24.50)=79.82, p<0.01). A corresponding t-test (Holm correction) as a sub-effect test was carried out, and a significant difference was observed at 99-percent confidence level for all the combinations of the three levels. More specifically, the degree of target event responses decreased as the degree of tracking difficulty increased, and had lower values in proportion to the degree of difficulty of the numerical tasks.

### 3.2 Eye Fixation-Related Potentials

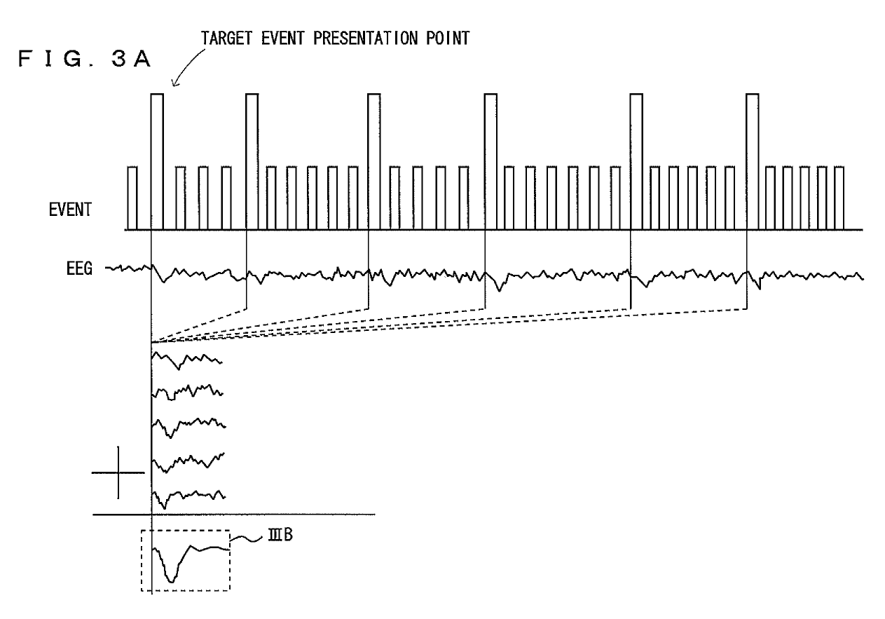

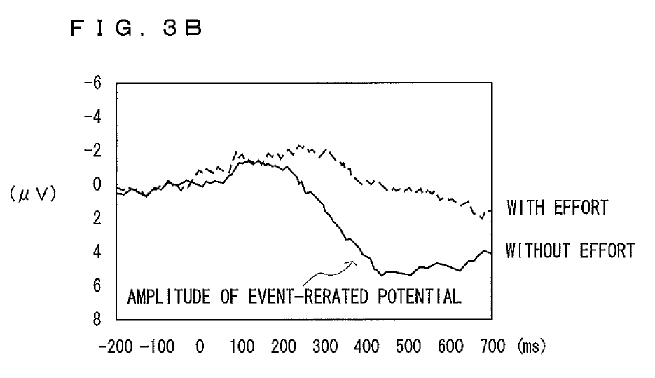

The total arithmetic means of the eye fixation-related potentials obtained from the backs of the heads (Oz) of all the subjects in the above-described conditions are given in FIG. 4. Among the eye fixation-related potentials, the positive component having a latency period of about 100 ms corresponds to a lambda response, and the peak value was obtained for each of the subjects. FIG. 5 shows the average amplitudes and their standard deviations in the conditions. As can be seen from FIG. 5, the lambda responses in conditions DE and DD in which a mental work load on the perceptual-motor system was imposed each had a decrease in the amplitude. Therefore, for the peak amplitude of each of the lambda responses, analysis of variance was applied to a two-factor within-subject design for a tracking task (two levels) and a numerical task (three levels). As a result, a main effect was observed in the tracking task (F(1, 17)=6.26, p<0.05). More specifically, the peak amplitude of the lambda response decreased as the degree of tracking difficulty increased. Note that there was no main effect in the numerical task and no interaction was observed.

3.3 Auditory p300

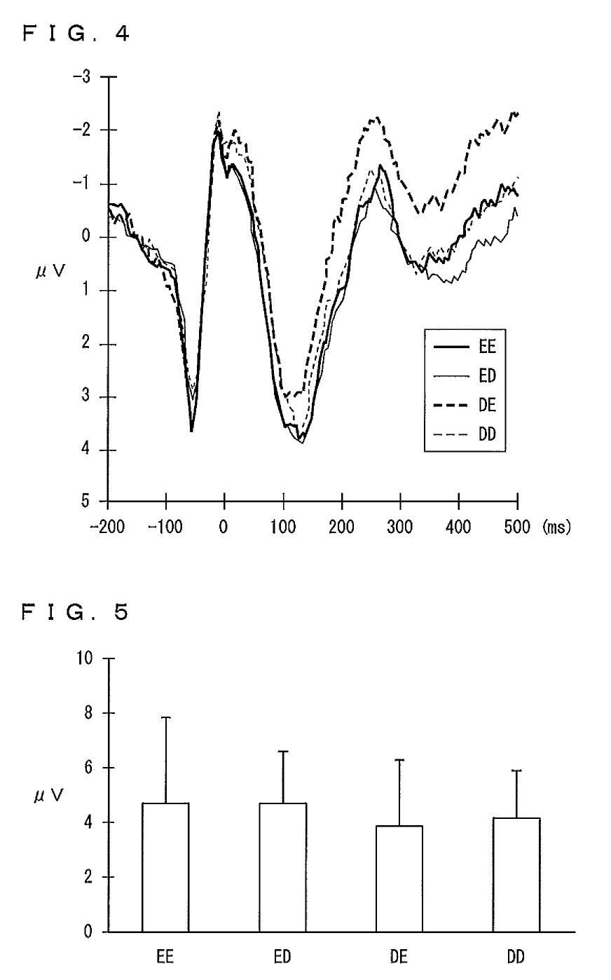

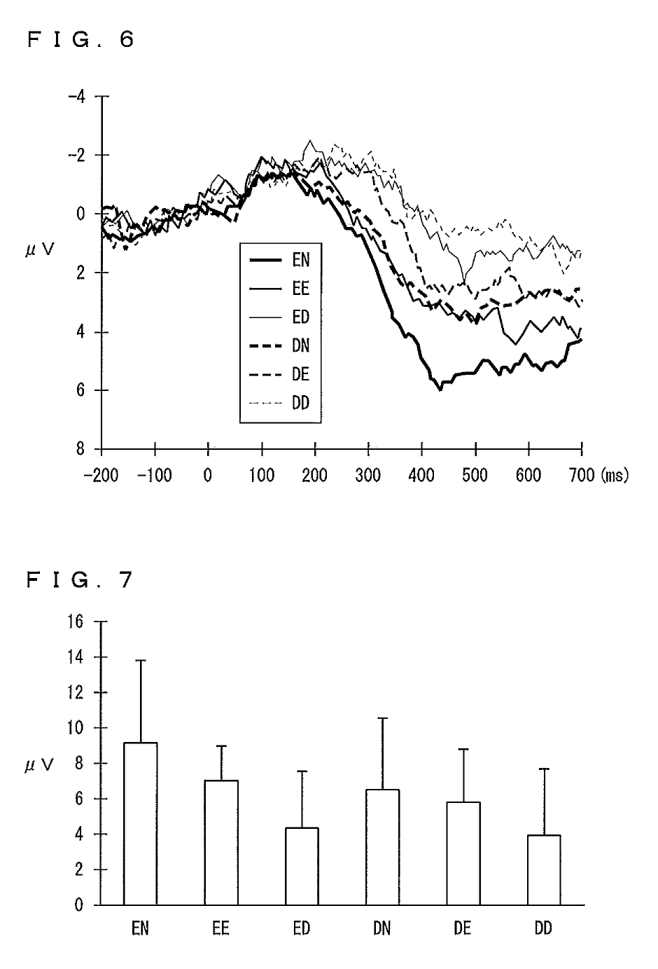

The total arithmetic means of auditory p300 in response to the target events obtained from the tops of the heads (Pz) of all the subjects in the above-described conditions are given in FIG. 6. In the arithmetic mean waveform, the maximum positive component in which a latent period was generated from 200 ms to 600 ms was determined as the auditory p300 and its peak value was obtained for each subject. FIG. 7 shows the average amplitude and the standard deviation in each of the conditions. As can be seen from FIG. 7, a reduction in the amplitude of the auditory p300 was observed in conditions EE, ED, DN, DE, and DD in which a mental work load on the perceptual-motor system and the perceptual-central processing system was imposed. Therefore, for the peak amplitude of the auditory p300, analysis of variance was applied to a two-factor within-subject design for a tracking task (two levels) and a numerical task (three levels). As a result, a main effect was observed in both the tracking task and the numerical task (the tracking task: F(1, 17)=11.53, p<0.01, the numerical task: F(1.25, 21.32)=9.27, p<0.01). A t-test (Holm correction) as a corresponding sub-effect test was carried out, and a significant difference was observed at 95-percent confidence level for all the combinations of the three levels. In other words, the peak amplitude of the auditory p300 was reduced in proportion to the degrees of difficulty of the tracking task and the numerical task. Note that no interaction was observed.

### 4. Analysis and Conclusion

As the result of subjective and the behavioral measures, step-wise difficulty degrees can be set by multiple tracking tasks, and mental work loads with the same degree of difficulty and different qualities (loads on the perceptual-motor system and the perceptual-central processing system) were imposed. The load on the perceptual-motor system was raised in level by changing the speed of the target in the tracking tasks. As a result, when the load is high, there were many tracking errors. The load on the perceptual-central processing system was raised in level by imposing the task of reading numbers aloud and additions in the numerical tasks. As a result, the number of utterance errors increased when the load was higher. The degree of subjective difficulty in each condition was divided into four levels (condition DD in the first level, conditions ED and DE in the second level, conditions EE and DN in the third level, and condition EN in the fourth level). Mental work loads with different qualities were imposed between conditions ED and DE and between conditions EE and DN, but no difference was observed in the degree of subjective difficulty. More specifically, the multiple tracking tasks employed in the experiments were believed to be suitable as tasks used to determine the effects of the difference in the degree of difficulty of tasks and the difference in quality among mental work loads. However, the degree of attention concentration increased in proportion to the degree of difficulty of the numerical task, while the degree of fatigue increased in the addition task, and therefore the difficulty alone was not necessarily controlled.

The effect of the perceptual-motor system loads on the event-related potentials was observed in both the peak amplitudes of the lambda responses and auditory p300. On the other hand, the effect of the perceptual-central processing system loads was observed only in the amplitude of auditory p300. From the result that a task load on the perceptual-motor system does not affect a lambda response, it would be difficult to utilize the lambda response to evaluate the degree of the load on the perceptual-central processing system. The peak amplitude of the auditory p300 also decreased in proportion to the load level of the numerical task. Consequently, the peak amplitude of auditory p300 should be an evaluation index used to totally evaluate the degree of the load on the perceptual-motor system and the perceptual-central processing system.

However, for the auditory p300, a stimulus must be presented or a response should be requested, so that it would be necessary to carry out signal processing such as independent component analysis by increasing the number of electrodes if there is too much eye movement. The lambda response can be measured while the eyes can be moved freely and therefore it is not necessary to present a stimulus or request a response. Therefore, it would be an effective index in consideration of its application to the load measurement of a driver. However, it cannot be measured in a condition without a saccade like that used in the experiments, and the load of the task on the perceptual-central processing system cannot be evaluated, so that it is necessary to examine in advance the content of an evaluation task when it is used as an evaluation index. In the present experiments, two kind of event-related potentials, i.e., the lambda response of an eye-fixation related potential and the auditory p300 were compared as indexes used to quantitatively evaluate the load of information processing on the user in human computer interaction in real time. It was found as a result that the peak amplitudes of these indexes both decreased due to the effect of the mental work loads, which showed that they were effective evaluation indexes. Furthermore, since the lambda response and auditory p300 responded differently to different mental work loads on the perceptual-motor system and the perceptual-central processing system, it was clearly demonstrated that these indexes did not have the same response characteristic. The lambda response may be an evaluation index for the perceptual-motor system and the auditory p300 may be an evaluation index for the perceptual-central processing system. Using the characteristics of these indexes, what kind of loads are upon a user can be specified by simultaneously measuring the lambda response and the auditory p300. When, for example, only the auditory p300 is used as an index, the loads on the perceptual-motor system and the perceptual-central processing system cannot be discriminated from each other, while the load on the perceptual-central processing system can be separately evaluated by simultaneously measuring the lambda response.

### First Preferred Embodiment

The following findings were obtained from the above-described experiments.

(1) As the mental work load on the perceptual-motor system increases, the peak amplitude of the lambda response decreases, while an increased mental work load upon the perceptual-central processing system does not reduce the peak amplitude of the lambda response.

(2) As a mental work load on the perceptual-motor system or the perceptual-central processing system increases, the peak amplitude of the auditory p300 decreases.

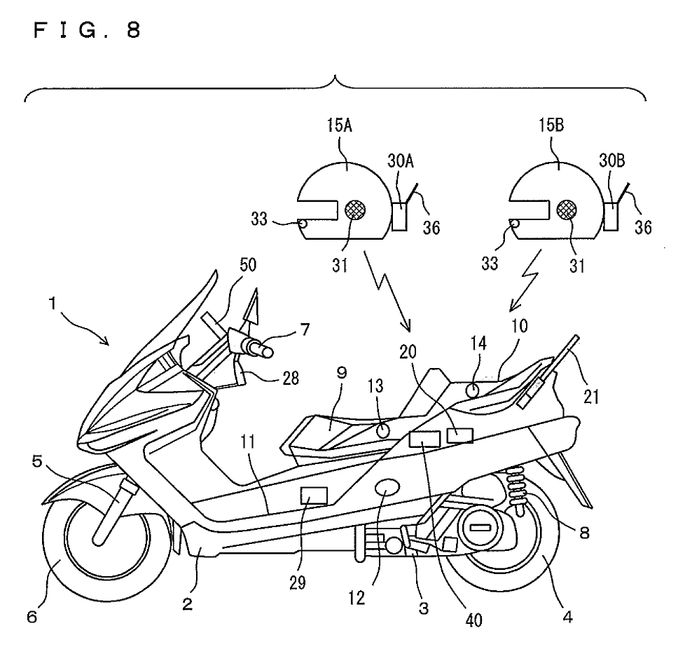

A first preferred embodiment of the present invention in the following is based on the above-described findings. FIG. 8 is a side view of the general structure of a motorcycle including a mental work load detector according to the first preferred embodiment of the present invention.

With reference to FIG. 8, the motorcycle 1 includes a mental work load detector 40, on-vehicle radio communication equipment 20, on-vehicle information equipment 50, a helmet side radio communication device 30A attached to a helmet 15A for a rider, and a helmet side radio communication device 30B attached to a helmet 15B for a passenger.

The motorcycle 1 includes a body frame 2, a power unit 3 attached to the body frame 2 so that the unit can swing up and down with respect to the body frame 2, a rear wheel 4 that receives driving power from the power unit 3 to rotate, a front wheel 6 as a steering wheel attached to the front of the body frame 2 through a front fork 5, and a handle 7 that integrally pivots with the front fork 5. The handle 7 is provided with a main power switch 28.

The power unit 3 is swingably coupled to the lower portion of the body frame 2 near the center of the frame and also elastically coupled to the rear portion of the body frame 2 through a rear suspension unit 8. At the top of the body frame 2 near the center, a seat 9 for a rider is provided and a seat 10 for a passenger is provided behind it. In the body frame 2, there is a rider step 11 for a rider to place his feet in a position between the seat 9 and the handle 7. Steps 12 for a passenger to place his feet are provided on both sides of the body frame 2 under the rider seat 9. There are a rider seat seating sensor 13 and a passenger seat seating sensor 14 on the seats 9 and 10, respectively that detect the sitting states of the rider and the passenger.

The on-vehicle communication device 20 is preferably fixed to the body frame 2 at a position below the passenger seat 10, for example. The on-vehicle communication device 20 is connected to an antenna 21 fixed to the body frame 2 behind the passenger seat 10 and carries out radio communication between the helmet side radio communication devices 30A and 30B. The on-vehicle information equipment 50 is preferably fixed to the handle 7 and further connected to the on-vehicle communication device 20 by wire, for example. Examples of the on-vehicle information equipment 50 include, for example, a navigation system that provides voice guidance for a traveling route, a music player, a radio, and a telephone voice relay that relays a voice sound from a mobile phone. The on-vehicle communication device 20 and the on-vehicle information equipment 50 receive power from an on-vehicle battery 29.

A pair of speakers 31 is fixed each at the inner surfaces of the helmet 15A and 15B facing the left and right ears of the rider and passenger, and a microphone 33 is fixed at a position facing each of the mouths of the rider and the passenger. The helmet side radio communication devices 30A and 30B are fixed at the backs of the helmet bodies. The helmet side radio communication devices 30A and 30B include antennas 36 and are connected to the speakers 31 and the microphones 33.

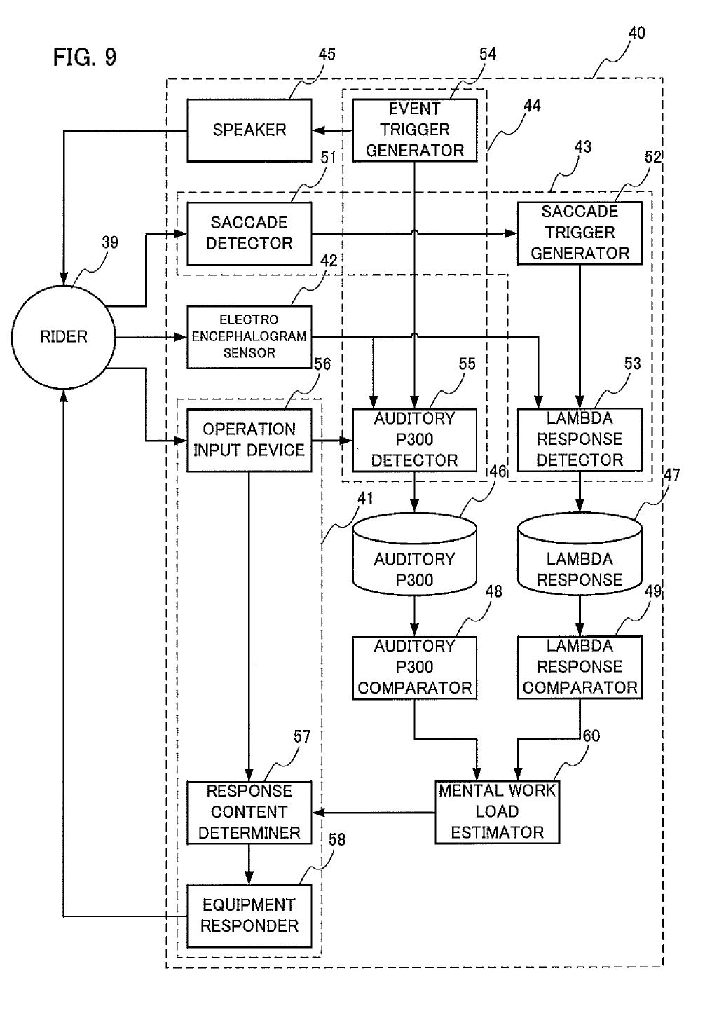

With reference to FIG. 9, the mental work load detector 40 detects the electroencephalogram of a rider 39 as a user that controls equipment 41, measures eye fixation-related potentials and event-related potentials, and estimates loads on the perceptual-motor system and the perceptual-central processing system of the rider 39.

More specifically, the mental work load detector 40 includes the equipment 41, an electroencephalogram sensor 42, an eye fixation-related potential measuring device 43, an event-related potential measuring device 44, a speaker 45, an auditory p300 database 46, a lambda response database 47, an auditory p300 comparator 48, a lambda response comparator 49, and a mental work load estimator 60. Here, the eye fixation-related potential measuring device 43, the event-related potential measuring device 44, the auditory p300 comparator 48, the lambda response comparator 49, and the mental work load estimator 60 are implemented by hardware resources by installing in a computer a mental work load detection program that will be described below.

The equipment 41 preferably includes devices unique to the motorcycle 1 (such as a meter panel, a handle, an accelerator, a brake, and a shifter) and on-vehicle information equipment 50. The rider 39 operates the motorcycle 1 by controlling the handle, the accelerator, the brake, the shifter and the like while keeping his attention on the road environment including other vehicles, pedestrians, signs, and the like. The rider 39 sometimes looks at the on-vehicle information equipment 50 while carrying out such basic operations.

The electroencephalogram sensor 42 senses the electroencephalogram of the rider 39. Therefore, sensor electrodes are attached at the back of the head (Oz) and the top of the head (Pz).

The eye fixation-related potential measuring device 43 includes a saccade detector 51, a saccade trigger generator 52, and a lambda response detector 53 and analyzes the electroencephalogram detected by the electroencephalogram sensor 42 to measure an eye fixation-related potential. The saccade detector 51 detects the saccade of the rider 39 based on an electrooculogram (EOG), for example. The saccade trigger generator 52 generates a trigger used to detect a lambda response at the ending point of the saccade detected by the saccade detector 51. The lambda response detector 53 detects a lambda response from the eye fixation-related potential about 100 ms after the ending point of the saccade in response to the trigger generated by the saccade trigger generator 52. More specifically, the eye fixation-related potential measuring device 43 analyzes in time series the electroencephalogram sensed by the electroencephalogram sensor 42 and detects a lambda response related to the mental work load on the perceptual-motor system.

The event-related potential measuring device 44 includes an event trigger generator 54 and an auditory p300 detector 55 and measures an event-related potential by analyzing the electroencephalogram sensed by the electroencephalogram sensor 42 in response to a sound (event) supplied to the rider 39 from the speaker 45. The event trigger generator 54 has the speaker 45 generate a sound to randomly generate a trigger used to detect auditory p300. The trigger may be generated according to the above described oddball task, for example, while the trigger may be generated during a timing in which the on-vehicle information equipment 50 generates a sound. The speaker 45 generates a sound in response to the trigger generated by the trigger generator 54 and transmits the sound to the rider 39. The speakers 31 provided inside the helmets 15A and 15B may serve the function of the speaker 45 instead. The auditory p300 detector 55 detects auditory p300 from the event-related potential about 300 ms after the ending point of the sound in response the trigger generated by the event trigger generator 54. More specifically, the event-related potential measuring device 44 analyzes in time series the electroencephalogram sensed by the electroencephalogram sensor 42 and detects auditory p300 related to mental work loads on the perceptual-motor system and the perceptual-central processing system.

The lambda response database 47 stores the data of the lambda response detected by the lambda response detector 53 in time series. The auditory p300 database 46 stores the data of auditory p300 detected by the auditory p300 detector 55 in time series.

The lambda response comparator 49 reads out the data of the lambda responses from the lambda response database 47 in time series, compares the lambda response being presently detected and the previously detected lambda response and determines whether the lambda response has decreased. The auditory p300 comparator 48 reads out the data of the auditory p300 from the auditory p300 database 46 in time series, compares the auditory p300 being presently detected and the previously detected auditory p300 and determines whether the auditory p300 has decreased.

The mental work load estimator 60 estimates the kind and load on the mental work imposed on the rider 39 based on the result of determination by the lambda response comparator 49 and the auditory p300 comparator 48. More specifically, it is determined whether the mental work load on the perceptual-central processing system has increased or no mental work load has increased.

The equipment 41 includes an operation input device 56, a response content determiner 57, and an equipment responder 58. The operation input device 56 receives the operation of the driver 39, and more specifically, the device preferably includes the handle, the accelerator, the brake, and the shifter of the motorcycle 1, the operation button or the panel of the on-vehicle information equipment 50, and the microphone 33 for a voice recognition navigation system. In this example, the auditory p300 detector 55 detects auditory p300 in response to operations received by the operation input device 56, but input to the auditory p300 detector 55 from the operation input device 56 may not be necessary. This is because the auditory p300 detector 55 can detect auditory p300 in response to the trigger generated by the event trigger generator 54. The response content determiner 57 determines the content of information to be presented by the equipment 41 or the timing therefor according to the kind and load of the mental work estimated by the mental work load estimator 60 or the operation mode of the equipment 41 operated by the rider 39. The equipment responder 58 presents the information based on the content or in the timing determined by the response content determiner 57 to the rider 39 or sets the equipment 41 to the operation mode determined by the response content determiner 57.

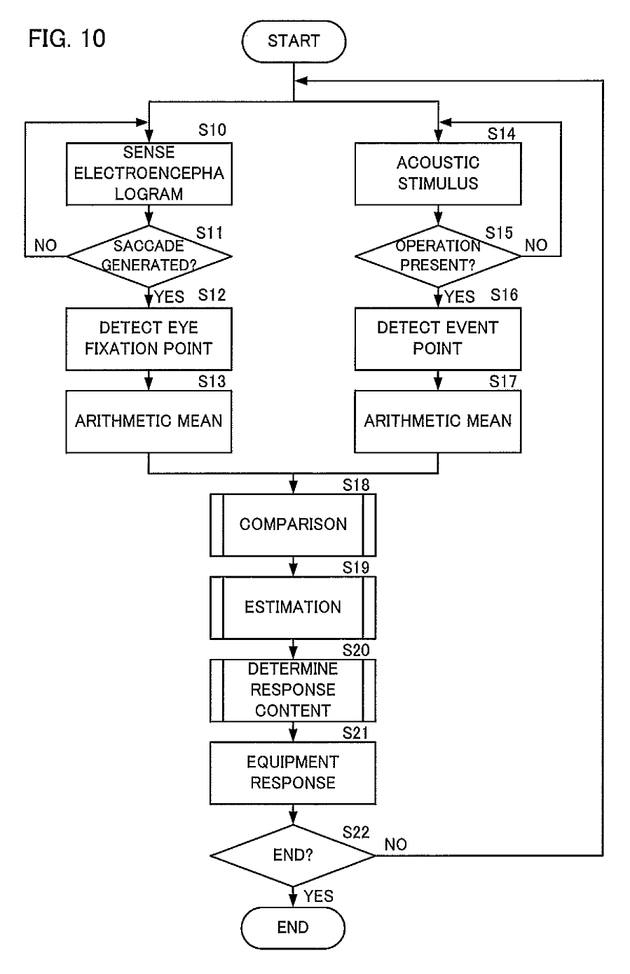

FIG. 10 is a flow chart for illustrating a mental work load detection program and the like that allow a computer to implement the functions of the mental work load detector 40. Now, with reference to FIG. 10, the operation of the mental work load detector 40 provided in the motorcycle 1 will be described.

The electroencephalogram sensor 42 senses the electroencephalogram of the rider 39 (S10). The saccade detector 51 detects the saccade of the rider 39 (S11), and the saccade trigger generator 52 generates a trigger if a saccade is generated (YES in S11). The lambda response detector 53 detects a lambda response appearing about 100 ms after the ending point of the saccade from eye fixation-related potentials included in the electroencephalogram in response to the trigger (S12), and stores the data in the lambda response database 47 in time series. The lambda response comparator 49 adds lambda responses and calculates the average (S13).

The event trigger generator 54 generates a trigger in parallel with the above-described process and the speaker 45 generates a sound in response to the trigger (S14). If the operation input device 56 receives the operation of the rider 39 (YES in S15), the auditory p300 detector 55 detects auditory p300 appearing about 300 ms from the point of the generation of the sound (presentation of the event) from event-related potentials included in the electroencephalogram in response to the trigger from the event trigger generator 54 (S16) and stores the data in the auditory p300 database 46 in time series. The auditory p300 comparator 48 adds auditory p300 and calculates the average (S17).

The lambda response comparator 49 then compares the average of the peak amplitude of the lambda response being presently detected and the average of the peak amplitude of the previously detected lambda response and determines whether the average of the peak amplitude of the lambda response has decreased (S18). The auditory p300 comparator 48 compares the peak amplitude of the auditory p300 being presently detected to the average of the peak amplitude of the previously detected auditory p300 and determines whether the average of the peak amplitude of the auditory p300 has decreased (S18). The mental work load estimator 60 estimates whether the mental work load on the perceptual-motor system has increased, the mental work load on the perceptual-central processing system has increased, or the mental work load on any of these systems has not increased in response to the result of determination by the lambda response comparator 49 and the auditory p300 comparator 48 (S19). The response content determiner 57 determines the content or timing of the information to be presented by the equipment 41 and the operation mode of the equipment 41 operated by the rider 39 in response to the result of estimation by the mental work load estimator 60 (S20). The equipment responder 58 then presents the information based on the content or in the timing determined by the response content determiner 57 to the rider 39 and sets the equipment 41 to the operation mode determined by the response content determiner 57 (S21). The above-described steps S10 to S21 are repeated unless an ending command is input (S22).

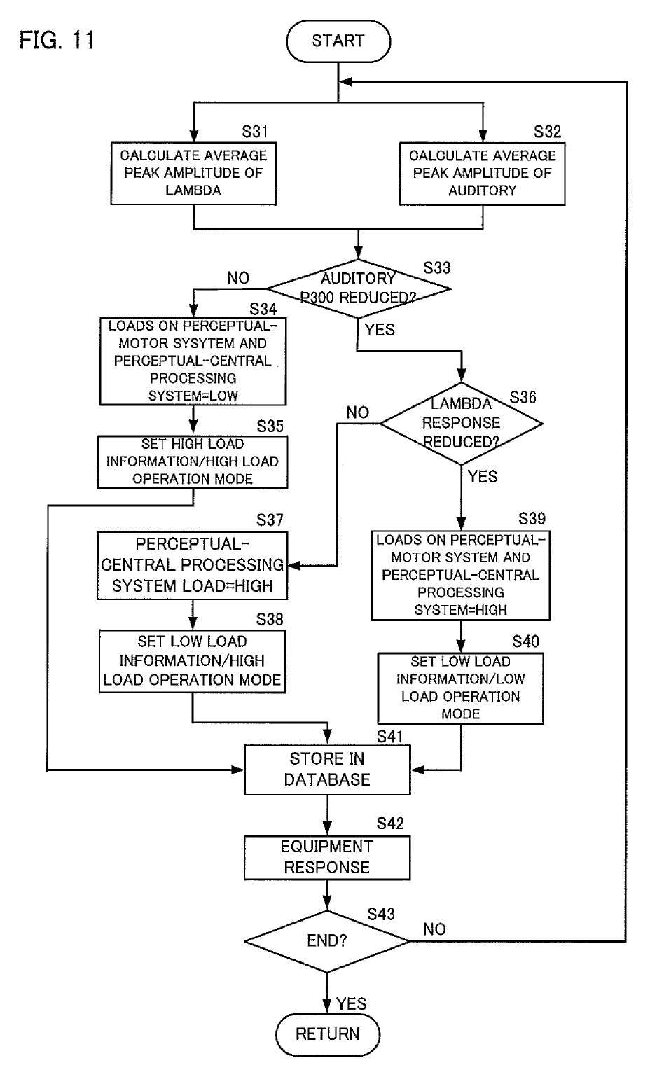

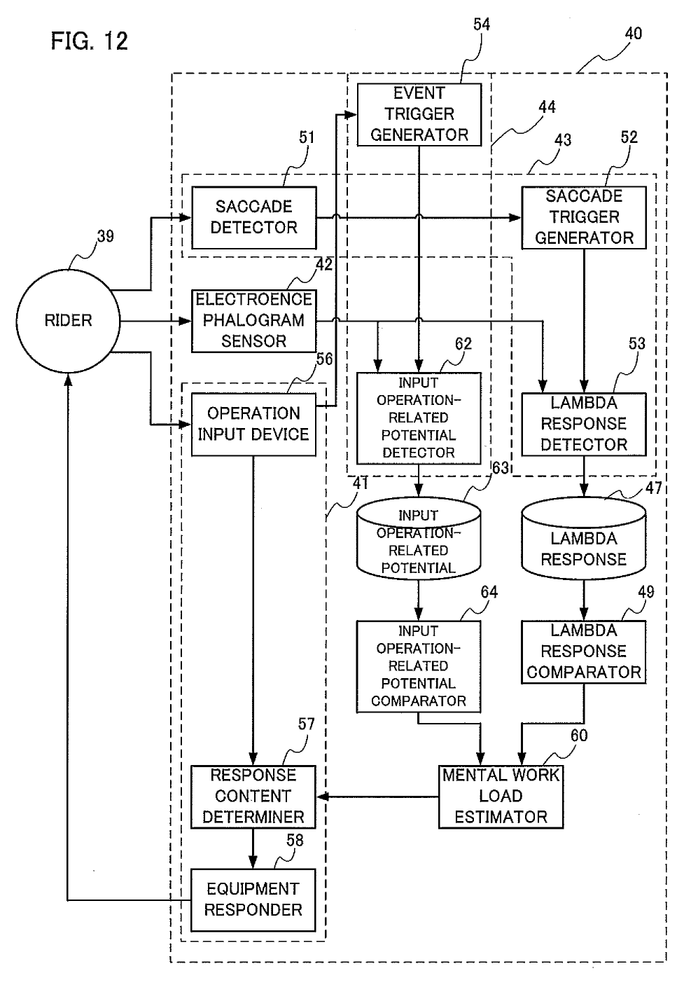

FIG. 11 shows details of the process from the comparison step S18 to the equipment response step S21 described above. With reference to FIG. 11, the average peak amplitude of the lambda response is calculated (S31) and the average peak amplitude of the auditory p300 is calculated (S32).

Then, if the average peak amplitude of the auditory p300 has not decreased (NO in S33), it is determined that the mental work load on both the perceptual-motor system and the perceptual-central processing system is low (S34). In this case, the equipment 41 is set to information and an operation mode with a high mental work load (S35). More specifically, the on-vehicle information equipment 50 is set to provide not only safety information but also music and entertainment information such as information about nearby facilities, and the motorcycle 1 is set to a sports mode for an experienced rider. The sports mode provides a higher output than the normal mode.

On the other hand, if the average peak amplitude of the auditory p300 has decreased (YES in S33) and the average peak amplitude of the lambda responses has not decreased (NO in S36), it is determined that the mental work load on the perceptual-central processing system is high and the mental work load on the perceptual-motor system is low (S37). In this case, the equipment 41 is set to information with a low mental work load and to an operation mode with a high mental work load (S38). More specifically, the on-vehicle information equipment 50 is set to present safety information only, and the motorcycle 1 is set to the sports mode for experienced riders.

If the average peak amplitude of the auditory p300 has decreased (YES in S33) and the average peak amplitude of the lambda response has also decreased (YES in S36), it is determined that the mental work load on both the perceptual-motor system and the perceptual-central processing system is high (S39). In this case, the equipment 41 is set to information and an operation mode with a low mental work load (S40). More specifically, the on-vehicle information equipment 50 is set to present safety information only and the motorcycle 1 is set to the normal operation mode for beginners.

Then, the set levels of the information and the load are stored in the databases 46 and 47 (S41). The equipment 41 responds to the set information and operation mode (S42). The above-described steps S31 to S42 are repeated unless an ending command is input (S43).

According to the first preferred embodiment, a lambda response related to a mental work load on the perceptual-motor system is detected from an eye fixation-related potential included in the electroencephalogram of a person and auditory p300 related to a mental work load on the perceptual-motor system and the perceptual-central processing system is detected from an event-related potential at the same time, so that the mental work load on the perceptual-motor system and the mental work load on the perceptual-central processing system can separately be estimated depending on whether the lambda response has decreased or whether the auditory p300 has decreased.

Furthermore, if both the mental work loads on the perceptual-motor system and the perceptual-central processing system are low, the equipment 41 is set to information and an operation mode with a high mental work load, and if the mental work load on the perceptual-central processing system is high but the mental work load on the perceptual-motor system is low, the equipment 41 is set to information with a low mental work load and an operation mode with a high mental work load. If the mental work loads on the perceptual-motor system and the perceptual-central processing system are both high, the equipment is set to information and an operation mode with a low mental work load, and therefore the information can be presented to the person based on an appropriate content and in an appropriate timing, so that the person can operate the equipment 41 in a suitable operation mode.

According to the first preferred embodiment, a sound is generated by the speaker 45 and the rider 39 is supplied with an acoustic stimulus, but the rider 39 may be given a visual stimulus instead from an image displayed on a display or light generated by a light emitting diode. In short, any stimulus recognizable via the human five senses may be provided.

### Second Preferred Embodiment

According to the first preferred embodiment described above, the rider 39 is given a sensory stimulus from the speaker 45 or the like and an event-related potential is measured in response to the stimulus. Instead of such giving a stimulus, the operation of the rider 39 may be determined with the operation input device 56 and an event-related potential in response to the operation (hereinafter referred to as “input operation related potential”) may be measured. In this case, an input operation related potential detector 62 may be provided instead of the auditory p300 detector 55 shown in FIG. 9, an input operation related potential database 63 may be provided instead of the auditory p300 database 46 shown in FIG. 9, and an input operation related potential comparator 64 may be provided instead of the auditory p300 48 shown in FIG. 9. The detector 62, the database 63, and the comparator 64 preferably have principally the same functions as those shown in FIG. 9.

The first and second preferred embodiments both refer to the examples in which the mental work load detector 40 is provided in the motorcycle 1, while the present invention is not limited to a motorcycle and may be provided in an automobile and other kinds of vehicles. Furthermore, the present invention is not limited to vehicles and may be provided in a ship and other kinds of vessels as well as a television, a video recorder, a mobile phone, a personal computer and other kinds of information equipment. Furthermore, the mental work load detector can be used simply as an evaluator for mental work loads without providing the response content determiner 57 and the equipment responder 58.

While preferred embodiments of the present invention have been described above, it is to be understood that variations and modifications will be apparent to those skilled in the art without departing the scope and spirit of the present invention. The scope of the present invention, therefore, is to be determined solely by the following claims.

What is claimed is:

1. A mental work load detector comprising:
an electroencephalogram sensor arranged to sense an electroencephalogram of a person;
a first biological reaction detector arranged to detect, in time series, a first biological reaction related to a first mental work load by analyzing the electroencephalogram sensed by the electroencephalogram sensor;
a second biological reaction detector arranged to detect, in time series, a second biological reaction related to a second mental work load different in quality from the first mental work load by analyzing the electroencephalogram sensed by the electroencephalogram sensor;
a first biological reaction comparator arranged to determine whether the first biological reaction detected by the first biological reaction detector has changed in time series;
a second biological reaction comparator arranged to determine whether the second biological reaction detected by the second biological reaction detector has changed in time series; and
a mental work load estimator arranged to estimate a kind and a load of a mental work imposed on the person based on a result of determinations performed by the first and second biological reaction comparators.

2. The mental work load detector according to claim 1, further comprising:
a sensory stimulator arranged to provide the person with a sensory stimulus; wherein
the first biological reaction detector includes an eye fixation-related potential measuring device arranged to measure an eye fixation-related potential; and
the second biological reaction detector includes an event-related potential measuring device arranged to measure an event-related potential in response to the stimulus provided by the sensory stimulator.

3. The mental work load detector according to claim 2, wherein:
the stimulus provided by the sensory stimulator is an acoustic stimulus;
the eye fixation-related potential measuring device includes a saccade detector arranged to detect a saccade of the person, and a lambda response detector arranged to detect a lambda response as the first biological reaction in response to the saccade detected by the saccade detector; and
the event-related potential measuring device includes an auditory p300 detector arranged to detect an auditory p300 as the second biological reaction in response to the acoustic stimulus provided by the sensory stimulator.

4. The mental work load detector according to claim 3, wherein:
the first biological reaction comparator includes a lambda response comparator arranged to determine whether the lambda response detected by the lambda response detector has decreased;
the second biological reaction comparator includes an auditory p300 comparator arranged to determine whether the auditory p300 detected by the auditory p300 detector has decreased; and
the mental work load estimator includes a determiner arranged to determine that the mental work loads on a perceptual-motor system and a perceptual-central processing system of the person have increased if the auditory p300 comparator determines that the auditory p300 has decreased and the lambda response comparator determines that the lambda response has decreased; and
a determiner arranged to determine that the mental work load on the perceptual-central processing system has increased if the auditory p300 comparator determines that the auditory p300 has decreased, and the lambda response comparator determines that the lambda response has not decreased.

5. The mental work load detector according to claim 1, further comprising:
an operation input device arranged to receive an operation of the person; wherein
the first biological reaction detector includes an eye fixation-related potential measuring device arranged to measure an eye fixation-related potential; and
the second biological reaction detector includes an event-related potential measuring device arranged to measure an event-related potential in response to the operation received by the operation input device.

6. The mental work load detector according to claim 1, further comprising:
a response content determiner arranged to determine a content or timing of information to be presented by equipment operated by the person in response to the kind and the load of the mental work estimated by the mental work load estimator, the information being perceived by the person; and
an equipment responder arranged to present the person with the information based on the content or during the timing determined by the response content determiner.

7. The mental work load detector according to claim 1, further comprising:
a response content determiner arranged to determine an operation mode of equipment operated by the person in response to the kind and load of the mental work estimated by the mental work load estimator; and
an equipment responder arranged to set the equipment to the operation mode determined by the response content determiner.

8. A motorcycle comprising:
a mental work load detector including:
an electroencephalogram sensor arranged to sense an electroencephalogram of a person;
a first biological reaction detector arranged to detect, in time series, a first biological reaction related to a first mental work load by analyzing the electroencephalogram sensed by the electroencephalogram sensor;
a second biological reaction detector arranged to detect, in time series, a second biological reaction related to a second mental work load different in quality from the first mental work load by analyzing the electroencephalogram sensed by the electroencephalogram sensor;
a first biological reaction comparator arranged to determine whether the first biological reaction detected by the first biological reaction detector has changed in time series;
a second biological reaction comparator arranged to determine whether the second biological reaction detected by the second biological reaction detector has changed in time series; and
a mental work load estimator arranged to estimate a kind and a load of a mental work imposed on the person based on a result of determinations performed by the first and second biological reaction comparators.

9. The motorcycle according to claim 8, further comprising:
a sensory stimulator arranged to provide the person with a sensory stimulus; wherein
the first biological reaction detector includes an eye fixation-related potential measuring device arranged to measure an eye fixation-related potential; and
the second biological reaction detector includes an event-related potential measuring device arranged to measure an event-related potential in response to the stimulus provided by the sensory stimulator.

10. The motorcycle according to claim 9, wherein:
the stimulus provided by the sensory stimulator is an acoustic stimulus;
the eye fixation-related potential measuring device includes a saccade detector arranged to detect a saccade of the person, and a lambda response detector arranged to detect a lambda response as the first biological reaction in response to the saccade detected by the saccade detector; and
the event-related potential measuring device includes an auditory p300 detector arranged to detect an auditory p300 as the second biological reaction in response to the acoustic stimulus provided by the sensory stimulator.

11. The motorcycle according to claim 10, wherein:
the first biological reaction comparator includes a lambda response comparator arranged to determine whether the lambda response detected by the lambda response detector has decreased;
the second biological reaction comparator includes an auditory p300 comparator arranged to determine whether the auditory p300 detected by the auditory p300 detector has decreased; and
the mental work load estimator includes a determiner arranged to determine that the mental work loads on a perceptual-motor system and a perceptual-central processing system of the person have increased if the auditory p300 comparator determines that the auditory p300 has decreased and the lambda response comparator determines that the lambda response has decreased; and
a determiner arranged to determine that the mental work load on the perceptual-central processing system has increased if the auditory p300 comparator determines that the auditory p300 has decreased, and the lambda response comparator determines that the lambda response has not decreased.

12. The motorcycle according to claim 8, further comprising:
an operation input device arranged to receive an operation of the person; wherein
the first biological reaction detector includes an eye fixation-related potential measuring device arranged to measure an eye fixation-related potential; and
the second biological reaction detector includes an event-related potential measuring device arranged to measure an event-related potential in response to the operation received by the operation input device.

13. The motorcycle according to claim 8, further comprising:
a response content determiner arranged to determine a content or timing of information to be presented by equipment operated by the person in response to the kind and the load of the mental work estimated by the mental work load estimator, the information being perceived by the person; and
an equipment responder arranged to present the person with the information based on the content or during the timing determined by the response content determiner.

14. The motorcycle according to claim 8, further comprising:
a response content determiner arranged to determine an operation mode of equipment operated by the person in response to the kind and load of the mental work estimated by the mental work load estimator; and
an equipment responder arranged to set the equipment to the operation mode determined by the response content determiner.
